(19) United States
(12) Patent Application Publication
(10) Pub. No.: US 2025/0163355 A1
(43) Pub. Date: May 22, 2025

(54) REACTOR FOR THE PRODUCTION OF GROWTH FACTORS TO REGENERATE THE EPITHELIUM OF THE AUDITORY SYSTEM FROM EMBRYONIC STEM CELLS, AIMED AT STIMULATING SENSORY TISSUE TO ENHANCE HEARING CAPACITY, IN THE FORM OF A LYOPHILIZED POWDER

(71) Applicant: Safa Samadzadeh Etehadi, Tehran (IR)

(72) Inventor: Safa Samadzadeh Etehadi, Tehran (IR)

(21) Appl. No.: 18/921,146

(22) Filed: Oct. 21, 2024

(51) Int. Cl. C12M 1/00; C12M 1/02; C12M 1/06; C12M 1/12; C12M 1/34; C12M 3/00

(52) U.S. Cl. CPC C12M 23/34; C12M 23/06; C12M 23/22; C12M 23/46; C12M 27/02; C12M 29/04; C12M 37/00; C12M 41/18; C12M 41/40; C12M 47/14

(57) ABSTRACT

The invention of a reactor for the production of growth factors to regenerate the epithelium of the auditory system from embryonic stem cells, aimed at stimulating sensory tissue to enhance hearing capacity, in the form of a lyophilized powder provides a novel reactor and method for producing growth factors that stimulate stem cells in the auditory region, promoting their conversion into auditory epithelial cells. The reactor comprises three sections: an upper transparent section for culture observation and sterilization via a UV lamp, a middle section for continuous mixing and purification using micro-filters, and a lower section for lyophilization. The reactor maintains optimal conditions for stem cell proliferation, utilizing controlled temperature and vacuum pressure to concentrate beneficial substances and crystallize growth factors into a lyophilized powder. This product serves as a potential treatment for auditory conditions, enhancing the regeneration of auditory structures and delaying the onset of presbycusis.

### TECHNICAL FIELD OF INVENTION

The present invention relates to a reactor and explains the method of using said reactor for the production of growth factors required to stimulate the epithelial tissue of the ear for the proliferation of middle ear epithelial tissue and the repair of auditory hair cells that would be achieved through the proliferation of stem cells derived from the umbilical cord of the fetus and the conversion of growth factors obtained from mesenchymal cells into a stable lyophilized powder and it also relates to a method for deactivating potential bacteria present in the growth factor production reactor using membrane filters equipped with nano-silver compounds.

### PRIOR ART

Epithelial cells, as one of the most important types of cells in the body, are found in various parts of the human body and perform a wide range of functions, including covering, protection, absorption, secretion, and sensing. These cells are present on the outer surface of the skin, lining the respiratory, digestive, and reproductive tracts, as well as in internal parts of some organs such as the heart and lungs. In fact, these cells create a protective layer against harmful agents and play a crucial role in maintaining the body's homeostasis. Additionally, epithelial cells in the ear have a specialized function in receiving sound and maintaining body balance.

Epithelial cells of the ear form part of the auditory and balance systems of the body and are distributed throughout the outer, middle, and inner ear. While the epithelial cells in the outer and middle ear primarily function as a protective barrier, the epithelial cells in the inner ear play a crucial role in transmitting sound and balance information to the central nervous system. These cells are specifically found in the cochlea and semicircular canals, and their role in processing auditory signals and balance-related information is essential.

Epithelial cells of the ear are generally divided into two categories: sensory and non-sensory epithelial cells. Sensory epithelial cells are mainly found in the inner ear and include inner and outer hair cells. Hair cells function as mechanical sensors that convert mechanical signals from sound and body position changes into electrical signals. The two main types of hair cells are as follows: Inner Hair Cells are arranged in single-cell rows along the cochlea, they are responsible for converting the mechanical energy from sound waves into electrical signals that are sent to the brain. Inner hair cells are primarily involved in the process of sound perception, and damage to these cells can lead to permanent deafness. Outer Hair Cells that are arranged in three rows along the cochlea, help amplify mechanical sound vibrations. They actively change their length to enhance auditory responses. This amplifying ability increases the ear's sensitivity to specific frequencies.

Non-sensory epithelial cells primarily play a supporting and protective role in the structures of the ear. Some of these cells are involved in supplying nutrients and regulating the ions necessary for the proper functioning of hair cells. Notable among the non-sensory cells are the epithelial cells of the inner wall of the cochlea, which contribute to maintaining the ionic environment required for transmitting nerve signals to the hair cells.

Another vital role of epithelial cells in the ear is maintaining body balance. Within the semicircular canals of the inner ear, sensory epithelial cells similar to hair cells are found, which help detect movement and changes in body position in space. These cells, along with organs such as the utricle and saccule, play a role in transmitting balance-related information to the brain.

The epithelial cells in the semicircular canals function as sensory cells, transmitting information related to head orientation and linear and angular acceleration to the central nervous system. This information is used to adjust body posture and eye movements. If these cells are damaged, balance disorders such as dizziness may occur.

Epithelial cells in the ear also serve as the first line of defense against external factors such as bacteria and viruses. These cells form a protective layer in the outer and middle ear, preventing infectious agents from entering the ear. Additionally, some epithelial cells in the middle ear are responsible for producing mucus and other protective substances that help eliminate invading agents.

The cellular structure of epithelial cells in the ear is designed to optimally perform the specific functions of each cell type. This structure varies among different types of sensory and non-sensory epithelial cells according to their functions. In this section, the cellular structure of each of these types is examined.

The cellular structure of hair cells has unique features that equip them for their function. One of the most prominent features is the presence of a set of microscopic structures called stereocilia, which extend from the upper surface of these cells. These thin, elongated structures bend in response to sound vibrations and mechanical movements, causing changes in the cell's electrical potential. These electrical changes are converted into nerve signals that are sent to the brain.

Hair cells have membranes that are specifically sensitive to the passage of potassium and calcium ions. These ions play a crucial role in the process of transmitting nerve signals. The membrane of hair cells responds to changes in pressure and vibrations, and with the entry of calcium ions, the release of neurotransmitters begins, which are then transmitted to the associated nerve cells.

Hair cells are located next to supporting cells that help provide stability and structural support. These supporting cells also play a role in maintaining the ionic environment around the hair cells, which is essential for their proper functioning.

Non-sensory epithelial cells primarily function in protecting, nourishing, and supporting hair cells. These cells possess structures that enable them to perform their tasks. Supporting cells act as physical support structures by creating a stable mechanical framework, helping to keep the hair cells in place. The structure of these cells mainly consists of a network of fibrous proteins such as actin and myosin, which enhance their resistance to mechanical stress and tension.

Non-sensory epithelial cells in the cochlea play a key role in maintaining the ionic balance of the inner ear. Through ion transport mechanisms, they preserve the environment surrounding the hair cells and prevent the depletion of essential ions. The membrane structure of these cells contains specific transport proteins that manage the exchange of ions such as potassium and sodium.

The epithelial cells of the outer and middle ear form a thin, protective layer over the ear surfaces. These cells have a simple structure that enables them to effectively perform their protective and secretory functions. Their structure consists of single-cell layers with smooth cell membranes, which act as a barrier to prevent the entry of pathogens and foreign substances into the ear. These cells are also capable of producing mucus, which aids in removing foreign particles.

In the ear's balance system, hair cells and supporting cells within the semicircular canals form specialized structures that help detect movements and changes in body position. In these cells, the stereocilia are connected to a gelatinous substance called the “cupula,” which shifts with head movement. The bending of the stereocilia within this gelatinous material aids in detecting motion and transmitting nerve signals. The cellular structure of these cells is similar to the hair cells found in the cochlea, with the key difference being that they are more sensitive to fluid movement within the canals rather than sound vibrations.

Damage to epithelial cells often leads to a reduction in hearing ability and causes hearing problems, such as ear heaviness, decreased signal production in the cochlear region, and an increase in auditory infections. The regeneration of the epithelial layer generally occurs very slowly, and in the case of auditory hair cells, there is almost no significant regrowth, making the damage to these cells a permanent condition. Breakage of the outer structures of auditory cells in the cochlea is one of the main factors leading to presbycusis and a subsequent decrease in sensitivity to specific frequencies.

In the past, the common treatment for individuals suffering from presbycusis or damage caused by typical injuries to the epithelial region of the ear was through the use of auditory amplification devices such as hearing aids. The amplification of sound waves by the hearing aid stimulates the auditory cells more intensely, allowing the affected individuals to hear sounds. However, over time, the damage tends to worsen, and hearing loss often progresses to complete deafness. Humanity has always sought new methods to alleviate these issues. Currently, research in reputable scientific sources indicates that the use of regenerative methods for damaged tissues to restore human abilities has replaced the use of assistive tools and devices. There is a growing preference for therapeutic approaches over control and assistive methods.

The term “stem cells” refers to cells that have a high capacity for division and can transform into cells of various parts of the body. During the process of proliferation, based on influencing factors within them, they can differentiate into specific and unique cell types. Generally, stem cells are found throughout the body; however, their quantity, type, and density can vary. The highest concentration of stem cells can be found during embryonic development and the early years of life. As the life cycle progresses, the number of these cells in body tissues decreases, to the point where the cell replication process reaches a minimal level.

Sources of stem cells include bone tissue, adipose tissue, teeth, brain, spinal cord, blood and blood vessels, skeletal muscle, skin and cornea, liver, pancreas, and the digestive system, as well as amniotic epithelial cells, placental stem cells, amniotic fluid stem cells, and umbilical cord stem cells. In embryonic stem cells, stem cells are extracted from the inner cell mass at the blastocyst stage. The use of embryonic stem cells is one of the most common methods for obtaining stem cell sources. Morphologically, at this stage, the blastocyst is where trophoblast cells, which form the placenta, are aggregated in a spherical mass, later developing into the placenta.

A Japanese invention with patent No. JP6979946B2 which was granted on 15 Dec. 2021 titled “How to Generate Human Inner Ear Sensory Epithelium and Sensory Neurons” relates to a method for producing sensory epithelium and sensory neurons of the human inner ear from human induced pluripotent stem cells. This method involves differentiating stem cells into inner ear sensory cells, including hair cells and sensory neurons, which aids in the regeneration and treatment of hearing loss or neural defects related to the inner ear. The main steps of this process include culturing pluripotent stem cells in specific environments containing inhibitors of signaling pathways, such as TGFβ and BMP. These cells are first differentiated into pre-otic epithelial cells, which subsequently develop into inner ear sensory cells. Additionally, the use of Wnt agonists and culturing in semi-solid media supports the formation of three-dimensional structures from the sensory cells. This invention is applicable for treating hearing loss through the transplantation of inner ear sensory cells produced from stem cells.

A Canadian invention with publication No. CA2823719A1 which was filed on 23 Jan. 2012 titled “Methods for generating inner ear cells in vitro” provides methods, compositions, and kits for generating inner ear cells under laboratory conditions. In the first stage, pluripotent stem cells are cultured in the presence of factors that suppress the formation of endoderm and mesoderm tissues, as well as factors that promote ectoderm growth, to produce a population of pre-placodal ectodermal cells. In the second stage, these cells are cultured under specific conditions with growth factors (FGF) to produce otic progenitor cells. In the third stage, these cells are cultured under specialized conditions to generate inner ear cells. The factors that suppress the formation of endoderm and mesoderm tissues include inhibitors of Wnt and TGFβ signaling. The suppressing or promoting ectoderm factors are used to activate IGF signaling. At various stages, otic progenitor and pre-placodal ectodermal cells may be enriched through mechanical methods.

A Chinese invention with publication No. CN109310713A which was filed on 2 Mar. 2017 titled “Make the controlled proliferation of vestibular stem cell/generation inner ear hair cells method using WNT and TGF-β inhibition” presents a method for the controlled proliferation of stem cells and the generation of inner ear hair cells by stimulating the WNT pathway and inhibiting the TGF-β pathway. This method aims to increase the number of supporting cells in the inner ear (such as Lgr5+ supporting cells) and convert them into inner ear hair cells. The supporting cells and hair cells produced through this method can be used to identify potential therapeutic compounds for treating hearing and balance loss. Moreover, this method can be applied to treat patients suffering from hearing or balance impairments who require the proliferation and differentiation of inner ear-supporting cells. Activators of the WNT pathway and inhibitors of GSK3 and TGF-β are used to efficiently proliferate and differentiate the supporting cells. This invention is highly beneficial for treating auditory and balance disorders caused by the reduction or loss of inner ear hair cells, and it can aid in restoring inner ear function.

A Chinese invention with patent No. CN111643671B which was granted on 30 Jul. 2021 titled “Composition for promoting hair cell regeneration and hearing recovery and application thereof” relates to a method for producing a compound to stimulate the regeneration of hair cells in the inner ear and improve hearing. The compound includes a Wnt agonist and one or more other inhibitors, such as VEGFR, Tgfbr, and ERG inhibitors. Through an organoid platform, this compound can facilitate the differentiation, maturation, and survival of hair cells in the inner ear. It can be used to develop medications aimed at regenerating inner ear hair cells and restoring hearing. Additionally, this method specifically addresses the prevention of premature hair cell death and enhances the efficiency of their regeneration. Using the organoid platform (a three-dimensional tissue model resembling the inner ear), the method promotes the differentiation of inner ear progenitor cells into hair cells. The compound not only accelerates hair cell regeneration but also reduces premature cell death, ensuring improved effectiveness of hearing-related therapies.

A Japanese invention with patent No. JP6486272B2 which was granted on 20 Mar. 2019 titled “Methods and compositions for hair cell and/or feeder cell regeneration” relates to methods and compounds for regenerating inner ear hair cells and feeder cells. By enhancing the activity of c-myc and Notch proteins, this invention induces the cell cycle and proliferation of inner ear hair cells. The regeneration of these cells can potentially improve hearing and the balance function of the inner ear. Additionally, feeder cells influenced by these methods differentiate into hair cells, thereby increasing the number of sensory cells in the inner ear. The simultaneous activation of c-myc and Notch proteins boosts cell cycle activity in inner ear cells. Once these proteins are activated, the cells effectively enter the proliferation phase, leading to an increase in hair cell numbers. These cells differentiate into hair cells, which can contribute to the restoration of hearing and balance. These methods may be applied to treat hearing loss caused by genetic or environmental factors, such as exposure to loud noise.

A Japanese invention with patent No. JP6961667B2 which was granted on 5 Nov. 2021 titled “Pathway for producing hair cells” describes a method and composition aimed at treating hearing loss caused by the loss of auditory hair cells in the inner ear. The method involves using specific compounds to enhance the expression and activity of the Atoh1 protein, which plays a crucial role in the differentiation of stem cells into auditory hair cells. Through the activation of the Wnt/β-catenin signaling pathway and the use of glycogen synthase kinase 3β (GSK3β) inhibitors and proteasome inhibitors, this method increases the number of hair cells in the inner ear. The primary application of this invention is for treating sensorineural hearing loss and auditory neuropathy caused by the loss of auditory hair cells. The compounds used include β-catenin polypeptides and Notch pathway inhibitors, which boost the number of auditory hair cells, leading to improved hearing function. This method can be administered locally or systemically through injections into the inner and middle ear.

A Korean invention with patent No. KR102493376B1 which was granted on 27 Jan. 2023 titled “Compositions, systems, and methods for generating inner ear hair cells for the treatment of hearing loss” presents methods and compositions designed to stimulate the self-renewal of stem/progenitor cells within the cochlear cell population. This method involves stimulating the proliferation of stem/progenitor cells while maintaining the ability of the daughter cells to differentiate into hair cells. Specifically, the invention relates to a biocompatible composition for treating or preventing hearing loss by targeting cochlear tissues. The composition includes two key components: CHIR99021 (or LY2090314) or its pharmaceutically acceptable salt, a small molecule widely recognized in stem cell research for promoting specific cell differentiation; and valproic acid or its pharmaceutically acceptable salt, commonly used in medical treatments such as epilepsy, but here utilized for its neuroprotective role and its ability to enhance cell survival. These active ingredients are embedded within a biocompatible matrix that allows for controlled release of the compounds over time. This matrix may consist of various materials, including hyaluronic acid, found in connective tissues; poloxamer, a polymer used in drug delivery systems; poly(lactic-co-glycolic acid) (PLGA); and collagen. A key feature of this formulation is its capability for the controlled release of active substances based on therapeutic needs.

A US invention with patent No. U.S. Pat. No. 9,896,658B2 which was granted on 20 Feb. 2018 titled “Generation of inner ear auditory hair cell” relates to an in vitro method for generating differentiated inner ear auditory hair cells, which are essential for sound transmission and hearing. The method is functionally divided into four key stages: MSCs are extracted from the bone marrow of mammals (such as humans). These cells have the potential to differentiate into various cell types and are used to produce inner ear auditory hair cells. The MSCs are cultured in a serum-free medium containing insulin-like growth factor-1 (IGF-1), epidermal growth factor (EGF), and basic fibroblast growth factor (bFGF). These growth factors promote the initial growth and proliferation of the stem cells. The stem cells are then cultured in a medium that includes neurotrophin-3 (NT-3) and brain-derived neurotrophic factor (BDNF). These compounds induce the cells to differentiate into auditory progenitor cells, which express proteins such as Sox2, Pax6, nestin, and Musashi, indicating their differentiation into neural cells. The progenitor cells are cultured for an appropriate period in the presence of gamma-secretase inhibitors. These inhibitors facilitate the final differentiation of the cells into inner ear auditory hair cells, which express specific hearing-related proteins, such as myosin VIIa and espin. Additionally, these differentiated cells may express one or both of the proteins atonal homolog 1 (Atoh1) and Jagged2. This method enables the production of differentiated auditory hair cells from stem cells, which can be used to treat hearing loss caused by damage to inner ear hair cells. The generated cells can also be transplanted into patients suffering from hearing loss due to neural damage, aiding in the restoration of hearing.

A US invention with patent No. U.S. Pat. No. 10,308,911B2 which was granted on Apr. 6, 2019 titled “Methods for generating the inner ear and other cranial placode-derived tissues using pluripotent stem cells” relates to a comprehensive method for generating pre-placodal ectoderm cells and inner ear sensory hair cells. This method uses pluripotent stem cells, which can differentiate into various types of cells in the body. In the first stage, human or mouse pluripotent stem cells are cultured for 1 to 2 days, forming embryoid bodies, which are embryonic-like structures. Proteins such as laminin, along with other proteins like entactin, type IV collagen, fibronectin, vitronectin, and a protein mix secreted by mouse sarcoma cells (EHS), are added to these embryoid bodies. The embryoid bodies are cultured for 2 to 3 days in the presence of proteins BMP2, BMP4, or BMP7 and a TGFβ inhibitor to induce differentiation into non-neural ectoderm. This non-neural ectoderm is then cultured for 3 to 5 days in a medium without BMP and TGFβ inhibitors but containing FGF and a BMP inhibitor, resulting in the formation of pre-placodal ectoderm cells. In the second stage, pre-placodal ectoderm cells are cultured for a specified period to convert them into otic placode cells. These otic placode cells express specific markers: in humans, they express Pax2, Pax8, and E-cadherin, while in mice, they express Pax2, Pax8, and Sox2. The differentiation into otic placode cells can be accelerated by the addition of a Wnt/β-catenin signaling activator, such as a Gsk3 inhibitor. In the final step, otic placode cells are cultured for a sufficient period in a specialized medium containing extracellular matrix proteins and N2 medium. This environment supports the final differentiation of the cells into inner ear sensory hair cells, which express specific proteins: in humans, they express MYO7A, BRN3C, and ATOH1, and in mice, they express MYO7A. This method provides a robust platform for producing sensory hair cells from pluripotent stem cells, which can be used for studying hearing loss and potentially treating conditions caused by the loss of inner ear hair cells.

A Spanish invention with patent No. ES2786925T3 which was granted on 14/10/2020 titled “Compositions and Procedures for Epithelial Stem Cell Growth and Expansion” provides a combined approach for repairing and enhancing epithelial stem cells in a damaged inner ear. By using a specific combination of a Wnt agonist and an HDAC inhibitor, this method enables the increase of LGR5-positive stem cells and promotes the regeneration of inner ear tissues. These characteristics make the drug combination highly applicable in various medical treatments, particularly in improving auditory function in individuals suffering from inner ear damage. Overall, this invention introduces an innovative pharmaceutical composition designed to enhance the proliferation of LGR5-positive epithelial stem cells in the inner ear's epithelial tissue, specifically for individuals with sensory hair cell damage in the Organ of Corti. The use of this combination leads to an increase in the proliferation of inner ear stem cells, facilitating the repair of damaged sensory hair cells. This composition is especially beneficial for individuals who have experienced damage to their sensory hair cells in the inner ear. By increasing the number of LGR5-positive epithelial stem cells, the composition aids in the regeneration and restoration of sensory hair cells, leading to improved auditory function.

A US invention U.S. Pat. No. 11,542,472B2 which was granted on Mar. 1, 2023 titled “Generation of inner ear cells” refers to a method for producing inner ear auditory hair cells from mammalian mesenchymal stem cells in an in vitro (laboratory) environment. In the first step, a population of mammalian mesenchymal stem cells is cultured in a serum-free medium containing insulin-like growth factor 1 (IGF-1), epidermal growth factor (EGF), and basic fibroblast growth factor (bFGF). This growth medium is critical for stimulating the cells to initiate the differentiation process. After the initial culture, the mesenchymal stem cells are placed in an environment containing neurotrophin-3 (NT-3) or brain-derived neurotrophic factor (BDNF). This medium induces the cells to differentiate into inner ear progenitor cells. These progenitor cells express the genes Sox2, Pax6, nestin, and Musashi, which are key markers indicating differentiation into inner ear hair cells. In the third stage, the inner ear progenitor cells expressing Sox2, Pax6, nestin, and Musashi are cultured in the presence of a gamma-secretase inhibitor (a chemical compound). The inhibitor is added to the medium in sufficient amounts and for an adequate duration to allow the cells to fully differentiate into auditory hair cells. Additionally, in one embodiment of the invention, the mesenchymal stem cells can be obtained from an individual who has experienced the loss of auditory hair cells due to sensorineural hearing loss.

An Australian invention with patent No. AU2015311816B2 which was granted on Apr. 4, 2019 titled “Compositions, systems, and methods for generating inner ear hair cells for the treatment of hearing loss” relates to methods and compositions for inducing the self-renewal of stem/progenitor supporting cells within the cochlear cell population, including inducing the proliferation of stem/progenitor cells while maintaining the daughter cells' capacity to differentiate into hair cells. One method for expanding the LGR5+ cell population in cochlear tissue with an existing LGR5+ cell population involves exposing the cochlear tissue to a composition that includes at least one stem cell proliferator. Additionally, at least one stem cell proliferator is capable of expanding the initial experimental population of LGR5+ cells in a stem cell proliferation assay to create an expanded experimental population. This expanded experimental population contains at least 10 times more LGR5+ cells than the initial experimental population. In this invention, one or more morphological features of the cochlear tissue are preserved. Furthermore, at least one stem cell proliferator is dispersed in a biocompatible matrix. The biocompatible matrix is a biocompatible gel or foam, and the composition is a controlled-release formulation. The cochlear tissue is living tissue within the body, and the stem cell proliferator includes at least one stem cell activator and one differentiation inhibitor.

An Australian invention with patent No. AU2017212655B2 which was granted on 18 Jan. 2024 titled “Expansion and differentiation of inner ear supporting cells and methods of use thereof” relates to methods for proliferating supporting cells of the inner ear (such as Lgr5+ supporting cells of the inner ear) and differentiating these supporting cells (like Lgr5+ supporting cells) into inner ear hair cells (such as Atoh1+ hair cells). It also covers the use of these supporting cells and hair cells, for example, in identifying potential therapeutic compounds to treat hearing and balance loss. Additionally, the described methods can be used to treat patients suffering from hearing and balance impairments, who would benefit from the enhanced proliferation and differentiation of inner ear supporting cells (like Lgr5+ supporting cells). The culture medium used in this invention includes the following components: a retinoid receptor signaling activator, a Wnt signaling activator, a bone morphogenetic protein (BMP) signaling inhibitor, a cyclin-dependent kinase activator, an E-box-dependent transcription activator, a Notch signaling activator, a histone deacetylase (HDAC) inhibitor, a proteolysis inhibitor, a PI3K-Akt signaling inhibitor, and a CREB protein activator. One or more of these factors are present in sufficient quantities to produce an expanded population of inner ear-supporting cells.

A US invention with publication No. US20050233448A1 which was filed on 16 Jun. 2004 titled “Materials and methods to produce stem cells” refers to a device for generating and culturing stem cells from a cell culture using perfusion (the flow of culture medium). The device consists of multiple membranes designed to attach stem cells. These membranes provide a surface for cell growth, and the stem cells adhere to them. The device also includes a mechanism for supplying the stem cells with a culture medium, delivering the necessary nutrients and growth factors to support cell growth. Additionally, it has a system for removing the used culture medium, which ensures that waste products are removed from the cellular environment, maintaining optimal conditions for cell growth. The device is designed so that the culture medium continuously flows through the stem cells. This flow aids in the better growth and differentiation of stem cells. The system for supplying and removing the culture medium is controlled by pumps, with the waste removal pump operating at a higher frequency than the supply pump. This improves nutrient exchange and waste elimination. The device can regulate the perfusion flow rate within a range of 0.05 to 5 milliliters per minute, which is optimized for stem cell growth conditions. Furthermore, the device is suitable for culturing various types of embryonic stem cells, including mammalian stem cells, mouse stem cells, and human stem cells.

A US invention with publication No. US20060024278A1 which was filed on 24 Jan. 2005 titled “Methods and products related to the production of inner ear hair cells” refers to a method for producing or regenerating inner ear hair cells, which can help improve a person's hearing or balance. According to this invention, the retinoblastoma protein (Rb) plays a critical role in keeping sensory cells out of the cell cycle and maintaining their non-dividing state. In the absence of pRb, inner ear hair cells continue to divide, and their differentiation occurs independently of pRb, a feature that aids in hair cell regeneration. In this method, by reducing or eliminating the expression of the retinoblastoma gene or the function of pRb, functional and differentiated inner ear hair cells can be produced or regenerated. This process can be performed both in vivo (within the body) and in vitro (outside the body). By reducing or eliminating pRb function in hair cells, supporting cells can also be regenerated. This regeneration is triggered by signals sent by the hair cells when pRb is reduced or eliminated. The method offers a way to regenerate functional inner ear hair cells and restore a person's hearing or balance through the reduction or elimination of pRb in the sensory cells of the inner ear. This method can be applied to patients who have experienced hearing loss due to various factors such as viral infections, noise-induced damage, genetic mutations, or ototoxic drugs. These approaches can also be used to regenerate nerve cells in both the central and peripheral nervous systems.

A US invention with publication No. US20060035375A1 which was filed on 16 Aug. 2004 titled “Method for selectively culturing epithelial or carcinoma cells” provides a method for the selective growth of epithelial or carcinoma (cancer) cells in vitro (under laboratory conditions). This method includes the following steps: a cell pellet containing digested epithelial or carcinoma cells is suspended in a primary growth medium that includes D-valine MEM, methylcellulose, serum, glutamine, and antibiotics. The concentration of methylcellulose in this medium is adjusted to inhibit the growth of fibroblast cells present in the pellet. The suspension is then added to a cell culture dish, the inner surface of which is at least partially coated with a medium containing protein extract, D-valine MEM, glutamine, and antibiotics. The suspension is incubated in the culture dish to allow for the selective growth of epithelial or carcinoma cells. The concentration of methylcellulose in the primary growth medium is adjusted to be within the range of 3.5 to 10 grams per liter. In some cases, the epithelial or carcinoma cells continue growing after being washed with a balanced salt solution and replacing the primary growth medium with a secondary growth medium that lacks methylcellulose. This method inhibits the growth of fibroblast cells, allowing for the selective growth of epithelial or carcinoma cells. In some cases, fibroblast cell growth is inhibited by up to 95%.

A US invention with publication No. US20200370007A1 which was filed on Oct. 8, 2020 titled “Methods of generating human inner ear sensory epithelia and sensory neurons” describes a method for producing a three-dimensional composition of human inner ear sensory tissue. According to this invention, pre-otic epithelial cells are embedded in a semi-solid culture medium that includes extracellular matrix proteins. These cells are derived from human pluripotent stem cells through the following steps: In the first stage, human pluripotent stem cells are cultured in a medium containing bone morphogenetic protein (BMP) and a small molecule inhibitor of TGFβ (transforming growth factor beta) signaling for approximately four days. In the second stage, the cultured cells from the first stage are further cultured for about four more days in the presence of fibroblast growth factor (FGF) and a BMP signaling inhibitor. In the third stage, the second-stage cells are exposed to a Wnt agonist for about four days, leading to the differentiation of the cells into pre-otic epithelial cells. These cells are then cultured for about 40 to 60 days in the presence of the Wnt agonist, creating conditions that allow the cells to spontaneously form otic vesicles (inner ear structures). The result of this process is the production of a three-dimensional composition of human inner ear sensory tissue.

A US invention with publication No. US20220204909A1 which was filed on 29 Dec. 2021 titled “Modular continuous flow bioreactor” relates to a modular cell bioreactor device designed for cell cultivation, consisting of various components. It includes multiple cell chambers positioned between an upper flow chamber and a lower flow chamber, several lower tubes connecting the lower flow chamber to one or more lower reservoirs, and several upper tubes connecting the upper flow chamber to one or more upper reservoirs. One or more pumps are connected to the reservoirs and upper and lower flow chambers through these tubes. Each cell chamber contains a lower permeable membrane in contact with the lower flow chamber, a three-dimensional distribution of cells, and an upper permeable membrane in contact with the upper flow chamber. The upper reservoirs may contain culture media, cell nutrients, cell supplements, buffering agents, oxygen or carbon dioxide gases, or antibiotics. The lower flow chamber and lower tubes may contain a buffer solution. The cell chamber can include a layer of epithelial cells placed on top of the three-dimensional distribution of cells, beneath the upper permeable membrane. The upper and lower tubes may have fluid or gas inlets and outlets, waste discharge ports, and sensors to measure temperature, pH, dissolved oxygen, or UV/Vis absorption.

An international invention with publication No. WO2008094597A2 which was filed in WIPO on 30 Jan. 2008 titled “Early mesoderm cells, a stable population of mesendoderm cells that has utility for generation of endoderm and mesoderm lineages and multipotent migratory cells (MMC)” relates to a method for differentiating primate pluripotent stem cells (pPSCs) into mesendoderm and mesoderm cells. The first step involves providing primate pluripotent stem cells (pPSCs), which can be derived from various sources, such as human embryonic stem cells (hESCs). In the next step, the pPSCs are exposed to an effective amount of a GSK inhibitor (such as BIO, TDZD-8, or other variants) in a cell differentiation medium for at least 18 hours to produce mesendoderm cells. Additionally, after the mesendoderm cells are produced, they can be separated or isolated for further use.

An international invention with publication No. WO2013166488A1 which was filed in WIPO on 6 May 2013 titled “Methods for generating the inner ear and other cranial placode-derived tissues using pluripotent stem cells” relates to a method for generating preplacodal ectoderm cells from pluripotent stem cells. In this method, pluripotent stem cells are first cultured under conditions that lead to the formation of embryoid bodies from these cells. Then, one or more extracellular matrix proteins are added to the embryoid bodies. The embryoid bodies are cultured in the presence of BMP2, BMP4, or BMP7 and a TGF inhibitor to form a non-neural ectoderm. The non-neural ectoderm formed in the previous step is then placed in a floating culture medium in the absence of BMP and the TGF inhibitor, and in the presence of an external FGF factor and a BMP inhibitor, to generate a cell population comprising pre-placodal ectoderm. In this process, the pre-placodal ectoderm cells are further developed into otic placodal cells, following similar steps. After the otic placodal cells are produced, they are converted into inner ear sensory hair cells. BMP4 and TGF inhibitors are used in this method to regulate the growth stages.

An Austrian invention with patent No. AU2007245453B2 which was granted on 28 Nov. 2013 titled “Substrate for the growth of cultured cells in three dimensions” is a cell culture substrate composed of a polymer emulsion with a highly polymerized internal phase provided, specifically designed and modified for the routine culture of cells in a three-dimensional environment, particularly mammalian cells. This substrate is used in cell culture systems to study and analyze cell proliferation, differentiation, and function. The cell culture substrate consists of multiple sections of a polymer emulsion with a highly polymerized internal phase, where the size of these sections ranges from 50 to 1000 micrometers. The pore volume of the polymer is between 88% to 92%, ideally around 90%. Additionally, in this invention, the cell culture container is a bioreactor designed to enhance the proliferation, differentiation, and function of the specified type of cell.

A US invention with patent No. U.S. Pat. No. 10,568,883B2 which was granted on 25 Feb. 2020 titled “Compositions, systems, and methods for generating inner ear hair cells for treatment of hearing loss” provides an advanced pharmaceutical composition, comprising a biocompatible matrix (such as poloxamer) in which specific active pharmaceutical agents, such as GSK3β inhibitors or Wnt agonists, Notch agonists, or HDAC inhibitors, are dispersed. This composition can be formulated for controlled release and can regulate drug release in various ways. Furthermore, the composition can be prepared in different forms, such as gel, foam, lyophilized, or hydrated, and includes a wide range of biocompatible materials for the matrix. These features make this pharmaceutical composition widely applicable in various medical treatments, particularly in regulating cellular signaling pathways and tissue repair.

A Chinese invention with patent No. CN201873686U which was granted on 22 Jun. 2011 titled “System capable of preparing stem cells by utilizing bioreactor and culture bottle/bag through continuous perfusion” relates to a device capable of systematically preparing stem cells using a bioreactor and a culture bottle/bag through continuous perfusion (constant infusion). This device comprises the following components: a reservoir for storing the culture solution, a monitored bioreactor, a central cell culture chamber, a waste/harvest fluid collection container, biochemical indicator detection units, an online monitoring system, and a sterile membrane filter. These components are interconnected through a piping system, with fluid flow rate controlled by a sterile fluid rate control device. The connections between the components are as follows: the culture solution reservoir is connected to the monitored bioreactor via the controllable piping system; the monitored bioreactor is connected to the input of the central cell culture chamber through the same piping system; the output of the central cell culture chamber is connected to the waste/harvest fluid collection container via the controllable piping system; the monitored bioreactor is connected to the online monitoring system through biochemical indicator detection units; and the online monitoring system is simultaneously connected to the biochemical indicator detection units for both the input and output of the central cell culture chamber.

### DESCRIPTION OF INVENTION

In the present invention, a special reactor and a specific method are used to produce growth factors that can stimulate stem cells in the auditory region and accelerate their conversion into auditory epithelial cells. The reactor consists of three sections stacked on top of each other. The upper section is made of a transparent material and has a circular shape, while the other two sections are made of 316L stainless steel. The top section is constructed from shatterproof glass (Pyrex) with a silicone-sealed lid, providing a defined volume to create the initial culture environment.

The upper section (FIG. 9, No. 203) of this reactor is designed from transparent glass to allow precise observation of ongoing processes. In this section, the culture medium containing stem cells along with growth factors are placed. This culture medium, along with Wharton's jelly, which is a valuable nutritional substance for stem cells, is added to the reactor. The primary purpose of designing this section is to provide an optimal environment for the rapid and efficient proliferation of stem cells.

Inside this glass vessel, a UV lamp (FIG. 9, No. 202) is installed. This lamp continuously emits ultraviolet rays, which play a crucial role in sterilizing the culture medium. The sterilization process ensures that any bacteria or microbial contaminants that might have infiltrated the solution are eliminated. As a result, the culture environment remains continuously free of microbial pollutants, which significantly enhances the quality and speed of cell proliferation.

One of the main challenges in such processes is the precise control of temperature. Surrounding the glass vessel, there are tube-shaped jackets (FIG. 3, No. 101) that have inlets and outlets for the circulation of water at a controlled temperature. Around these tubes, a protective cover (FIG. 9, No. 215) is installed to enclose them. Temperature-controlled water flows steadily through these tubes, ensuring the vessel's internal temperature is consistently maintained at 4° C. This controlled temperature is designed to prevent any unwanted temperature fluctuations that could negatively affect cell proliferation and the performance of growth factors.

For precise temperature control, a thermal unit (FIG. 3, No. 103 and 105) is connected to the tubes. This unit automatically regulates the temperature and prevents any fluctuations. As a result, the culture medium is maintained under optimal conditions for cell growth and proliferation, ensuring that the proliferation process occurs in a stable and predictable environment.

The stem cell culture medium remains in this state for a period of 20 to 30 days. During this time, the cells rapidly divide and proliferate into billions of cells. This proliferation process occurs under optimal conditions, made possible by continuous sterilization from the UV lamp and precise temperature control. One of the advantages of this design is the ability to create a closed and protected environment, which prevents the infiltration of contaminants and bacteria into the culture medium.

The different sections of the reactor are connected using locks and levers (FIG. 9, No. 207). These locks are designed to securely hold the sections in place, preventing any leakage or escape of materials. After preparation and vacuum sealing, the culture medium begins the proliferation process, lasting 20 to 30 days. This duration is essential for the stem cells to proliferate into billions of cells. The reactor's multi-layered structure enables better management and processing of materials, allowing the user to simultaneously control different stages of the process.

A key feature of this invention is the presence of micro-filters between the sections (FIG. 9, Nos. 204, 205, and 206). These filters are responsible for separating unwanted materials and impurities from the culture medium. There are three filters, arranged from coarse mesh to fine mesh at the outlet passage. Above these filters, a lever (FIG. 8, FIG. 9, No. 218) is installed, allowing the filters to be opened and closed. The lever mechanism is designed so that by turning it 90 degrees to the right, the solution is directed from the upper section toward the filters. After passing through the filters, the purified materials are transferred to the next section. This mechanism ensures high precision and control in the filtration process, preventing any contamination or impurities.

In the middle section of the reactor (FIG. 9, No. 216), a rotating magnet (FIG. 9, No. 217), similar to a magnetic stirrer, is installed. This magnet acts as a stirrer, continuously mixing the culture medium while the solution is being transferred to the lower tank. The stirring action helps to homogenize the solution, detaching cells that have adhered to the walls of the vessel and returning them to the solution. This process improves the quality of cell proliferation and enhances operational efficiency.

By turning the lever 90 degrees, the solution is directed towards the filters, and after passing through them, it returns to the middle section. This continuous filtration and transfer process ensures that the culture medium remains consistently fresh and purified, playing a crucial role in preventing excessive cell density and maintaining the solution's balance.

Once the solution enters the middle section, a vacuum pump (FIG. 3, No. 102) is in place to lower the ambient pressure. This pressure reduction causes the water in the culture medium to begin evaporating at a temperature below 100° C. This evaporation process separates excess water from the solution, resulting in a higher concentration of beneficial substances within the medium. Concentrating the solution is vital for enhancing the quality of growth factors and increasing the density of stem cells.

In the final stage, by turning the lever again 90 degrees, the concentrated solution is transferred to the lower section (FIG. 9, No. 210). This section is equipped with a freezing system (FIG. 9, Nos. 209 and 214) that lowers the temperature to −80° C. This temperature reduction causes the growth factors present in the solution to crystallize into a powder form. Additionally, another vacuum pump (FIG. 3, No. 104) is located in this section to facilitate the powdering process.

The bottom of the lower section is designed with a gentle slope leading downward. At the lowest point of the vessel, a concave chamber (FIG. 4) is positioned to collect the powders generated in this area. This engineered design allows the user to easily extract the final product without the need for complex extraction procedures. The invention also includes a chassis (FIG. 9, No. 211) on which the equipment is mounted, with a cover (FIG. 9, No. 212) and a lid (FIG. 9, No. 213) that encloses the equipment.

To extract stem cells, the umbilical cord of a newborn, obtained through cesarean section, is first placed in physiological serum. After transferring the necessary number of umbilical cords from multiple newborns into the hood, the washing and sterilization process is performed using 70% ethanol, and the sterilized umbilical cords are cut into smaller pieces. After washing with PBS (phosphate-buffered saline) and placing the pieces in HBSS (Hank's Balanced Salt Solution), the washing process is completed. Next, in a completely sterile environment, the segmented umbilical cord pieces are cut open, and the Wharton's jelly is collected from them. For culturing stem cells, the upper section of the reactor is first filled with a sterile medium consisting of fetal bovine serum (FBS) and DMEM culture medium. Antibiotics such as streptomycin, gentamicin, and penicillin are added to prepare the culture medium in the upper section of the reactor. Additionally, enzymes like trypsin and buffers such as PBS are added to the solution to create optimal conditions for cell culture.

Research in previous articles and patents indicates that mesenchymal stem cells are isolated from three relatively distinct regions within Wharton's jelly: the pre-vascular region, the inter-vascular region, and the sub-amniotic region. Stem cells from umbilical cord blood and matrix are intermediate differentiated cells, between embryonic and fully mature cells. Wharton's jelly in the umbilical cord consists of two parts: connective tissue derived from the extraembryonic mesoderm and fibroblast-like cells. This cell population collectively forms umbilical cord mesenchymal stem cells, which have the potential for unlimited proliferation and the ability to differentiate into various tissues.

In the culture serum, by adding inhibitors of signaling pathways such as BMP (bone morphogenetic protein) and TGF-β (transforming growth factor beta-cytokine secreted by white blood cells), the stem cells are directed to differentiate into pre-otic epithelial cells, which subsequently develop into sensory cells of the inner ear. Additionally, the use of Wnt agonists can drive the process towards the formation of pluripotent auditory cells.

The presence of endoderm and mesoderm suppressors, along with at least one ectoderm re-stabilizing factor, leads to the production of a population of ectodermal cells. Fibroblast growth factor (FGF) assists in cultivating the population of pre-otic progenitor cells. This helps support the LGR5+ cell population in the inner ear, facilitating their conversion into auditory hair cells. Inhibitors of GSK3 (glycogen synthase kinase 3) help regulate the cell growth process and drive the conversion of basal cells into auditory hair cells.

When Wharton's jelly is placed in the upper section of the reactor, the necessary amounts of amino acids, vitamins, minerals, trace elements, and nucleotides are added to the system. Fetal bovine serum (FBS) is used due to its high levels of growth factors, abundant proteins, and ability to promote cell adhesion to the culture vessel surface. The supplements added to the medium, typically in nanogram quantities, generally include hepatocyte growth factor (HGF), platelet-derived growth factor AA, stem cell factor, insulin-like growth factor 1 (IGF-1), and fibroblasts. The use of these supplements enhances the growth, proliferation, and viability of mesenchymal cells compared to culture mediums without supplements. The growth factors are primarily mitogenic, promoting mitotic division in mesenchymal cells.

In environments without supplements, cells generally proliferate slowly and may adopt a flattened morphology. However, in supplemented environments, cells develop with higher density and sufficient elongation. The slender, spindle-shaped proliferating cells lead to greater cell crowding and higher density. Slow cell proliferation leads to gaps between the cells, contributing to their spherical shape and promoting premature cellular aging.

Before placing the Wharton's jelly, the medium is prepared by adding sodium bicarbonate powder, and a very low concentration of HCL is used to adjust the pH to a range of 2-7 to 4-7. A UV lamp installed on the upper lid of the reactor is used to sterilize the solution, with the lamp being turned on for 20 to 30 minutes to ensure complete sterilization. This environment is considered the base culture medium, and its temperature is maintained between 4 to 7° C., regulated by a water circulation system in the jacket surrounding the reactor.

After a period of 20 days to 1 month, by injecting PBS and sterile distilled water along with the enzyme trypsin, the glycoprotein bonds formed between the generated cells and the surface of the container are broken. The addition of the trypsin enzyme breaks the amino bonds in proteins, and after the introduction of the trypsin amino acid, the cells become detached. A covered magnet inside the upper section of the reactor, along with an electrically magnetizable rotor placed between the upper and middle sections, allows for the complete homogenization of the culture solution. Once sufficient cell proliferation and detachment occur in the glass section of the reactor, the culture medium containing the created cells and the secreted factors are thoroughly mixed, ensuring maximum extraction of growth factors from the generated epithelial cells in the solution.

The growth factors produced in the culture medium contain various growth factors and cytokines secreted by the stem cells. These include growth factors such as VEGF, PDGF, EGF, HGF, NGF, BDNF, IGF-I, IGF-II, KGF, PFGF, IL-6, and other growth factors, all of which are secreted by the stem cells during cell proliferation.

In this stage, by opening the bottom valve of the upper section of the reactor, the contents of the cell proliferation chamber pass through the lower outlet. As the solution passes through the filters in the drainage section, all cultured cells along with their DNA components remain behind the filters. The culture medium, along with the growth factors, passes through the filters and enters the second section of the reactor.

The new environment is a conditional medium, and since this conditional medium is free of biological cells or cell components due to filtration, injecting this solution into the body minimizes or completely eliminates inflammatory and immune responses in the recipient. This also reduces or eliminates immune reactions when transplanting parts or sections into the auditory system of the patient.

In this stage, the conditional medium in the second section is subjected to negative pressure through a vacuum pump. Over time, at room temperature, the water volume in the medium decreases. Once the desired concentration is reached, the concentrated solution is gradually passed through ultra-fine mesh filters coated with nanosilver and transferred to the lower section. This lower section functions as a freeze-dryer designed to produce lyophilized powder from the stem cell conditional medium.

The temperature of the lower section, which functions as a freezer, is reduced to −80° C., while simultaneously, the pressure in the lyophilized powder production section is decreased to 0.1 millibars. Ultimately, at the end of the process, this pressure is reduced to 0.03 millibar. This process takes between 24 to 48 hours, depending on the volume of the reactor. At the end, the powder collected in the concave section of the freeze-dryer consists of a lyophilized powder from the conditioned medium, to which liquid additives can be added for enrichment and for injection purposes when needed.

Using specific injection methods, the solution derived from the lyophilized powder containing epithelial growth factors can be injected into the middle and inner ear. This stimulates the proliferation of stem cells present in the auditory tissues of the patient. Along with the administration of essential vitamins and amino acids to the patient, the necessary materials reach the auditory region through the circulatory system. The presence of a colony of growth factors accelerates the proliferation of stem cells and rejuvenates the damaged surface epithelial tissues as well as the auditory hair cells in the hearing system.

Since the production of lyophilized powders in laboratory settings requires frequent transfers from culture containers to various environments, there is a risk of bacterial or viral contamination, and sometimes environmental factors can disrupt cell proliferation or direct it toward producing other types of cells in the body. Therefore, controlling the cell proliferation process in the present reactor ensures that a closed-loop, integrated system can produce the required lyophilized powder for cell stimulation in a sterile and completely safe manner. Additionally, by increasing the reactor volume or using multiple identical reactors in the production environment, a sufficient amount of medication can be prepared to meet the needs of all individuals requiring auditory repair.

The product generated in this reactor can serve as an epithelial growth supplement and a cell proliferation accelerator for auditory structures. It offers a potential method for treating presbycusis and regenerating the epithelial cilia in the auditory system. Availability of this product from the reactor could delay the need for hearing aids in elderly individuals due to presbycusis, and with proper use of the cellular growth stimulants during a patient's life cycle, it could prevent presbycusis or other auditory issues.

After obtaining the necessary approvals from the Food and Drug Administration, passing clinical tests, and receiving the required licenses, the present solution could be administered as ear drops into the external ear. This would promote the growth of epithelial cells in the outer ear, which are responsible for protecting this area. It could be especially beneficial for individuals who suffer from reduced earwax secretion or damage to the epithelial cells in this region.

### BRIEF DESCRIPTION OF FIGURES

FIG. 1 shows the overview of the front of the reactor.

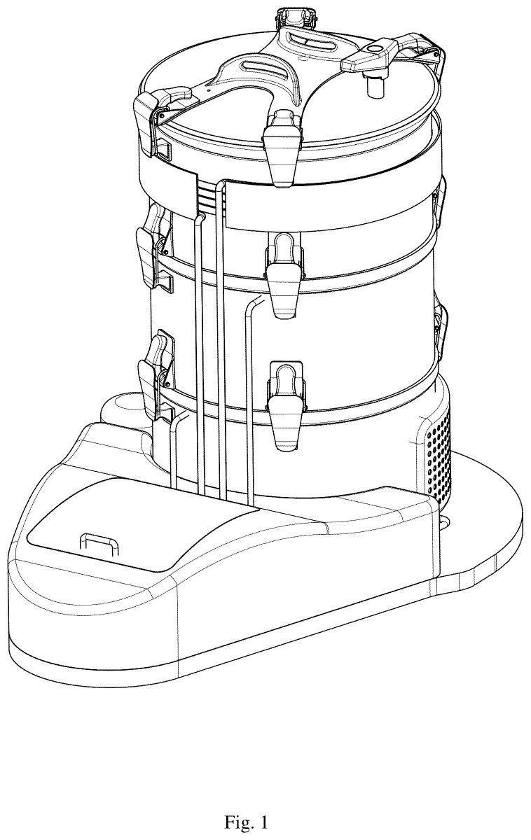

FIG. 2 shows the overview of the back of the reactor.

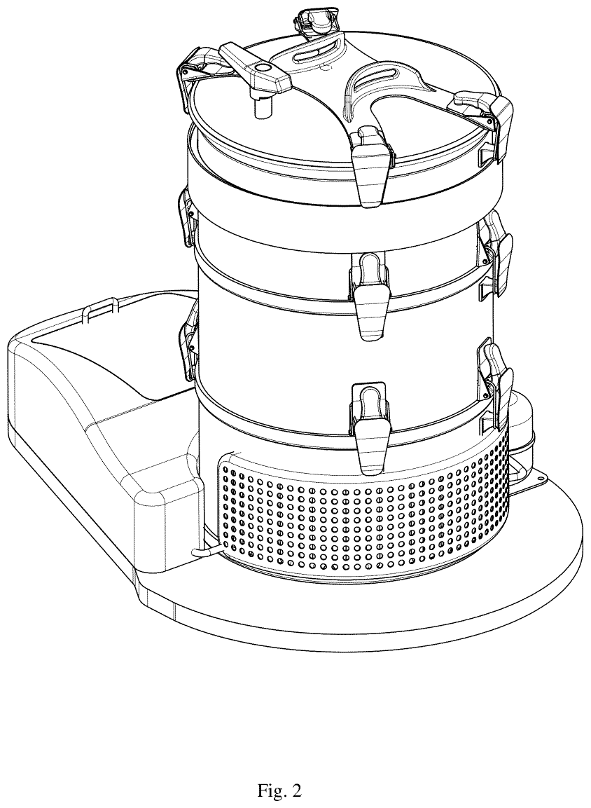

FIG. 3 shows the internal view of the equipment of the reactor.

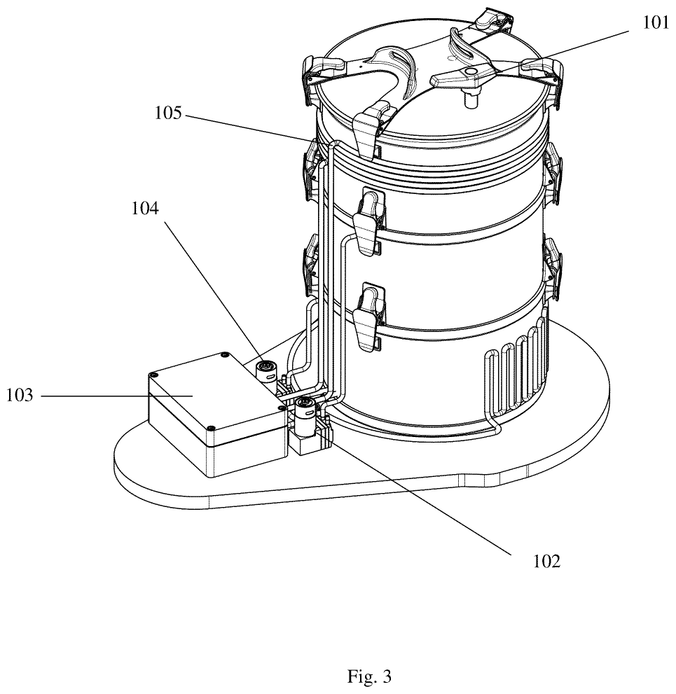

FIG. 4 shows a view of the lower section of the reactor.

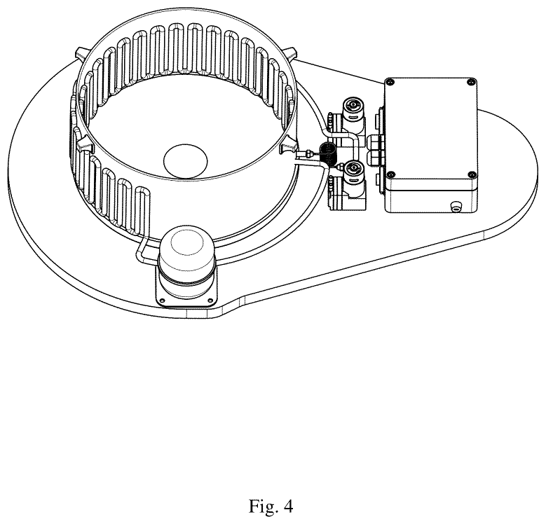

FIG. 5 shows a view of the middle section of the invention, along with the lever for opening and closing it.

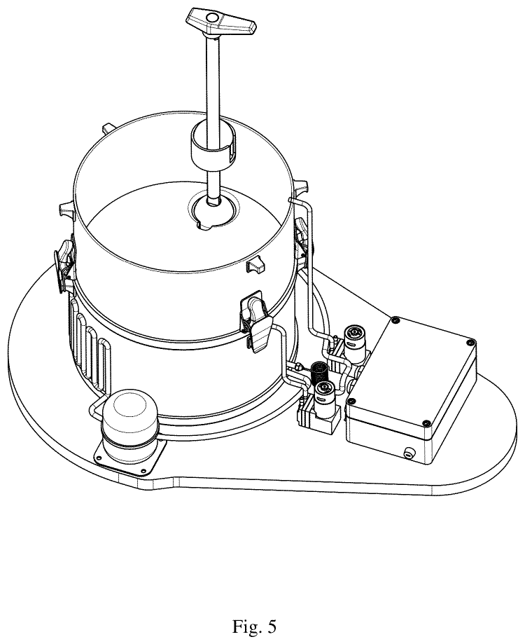

FIG. 6 shows a view of the upper section of the invention, along with the lever for opening and closing it.

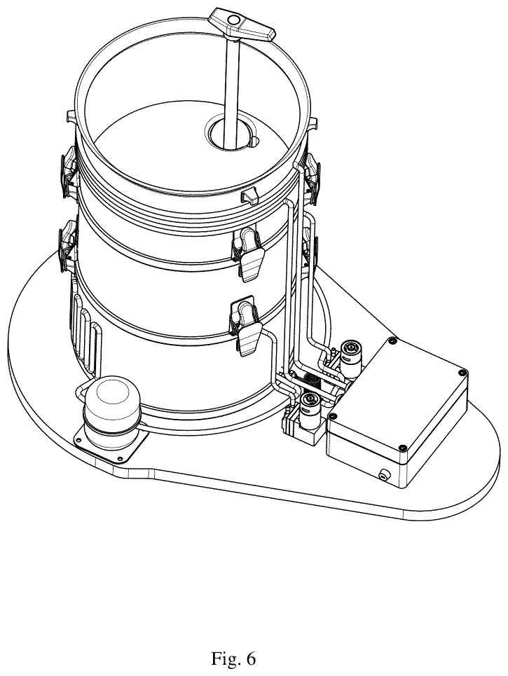

FIG. 7 shows a view of the three sections of the invention, along with the associated equipment.

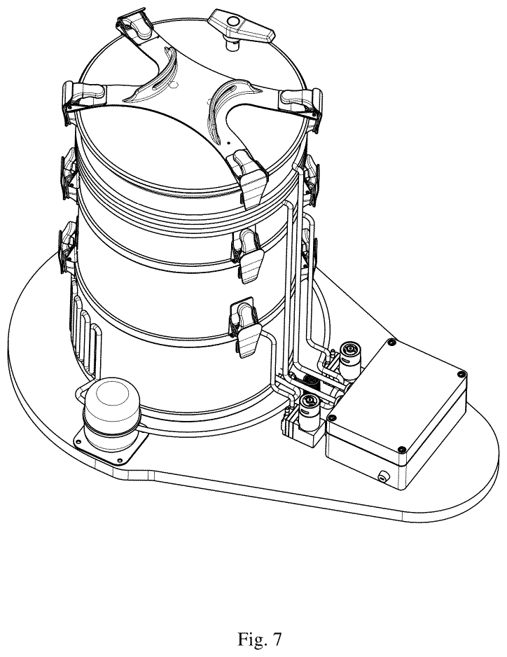

FIG. 8 shows an exploded view of the lever for opening and closing the sections.

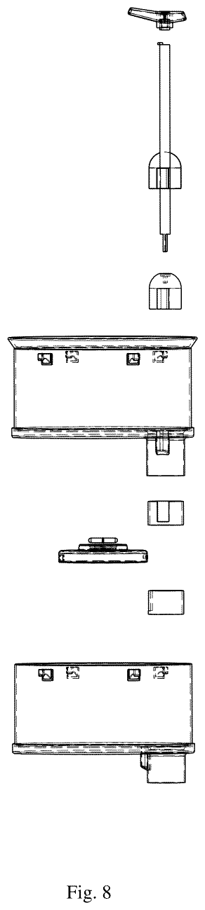

FIG. 9 shows an exploded view of the reactor.

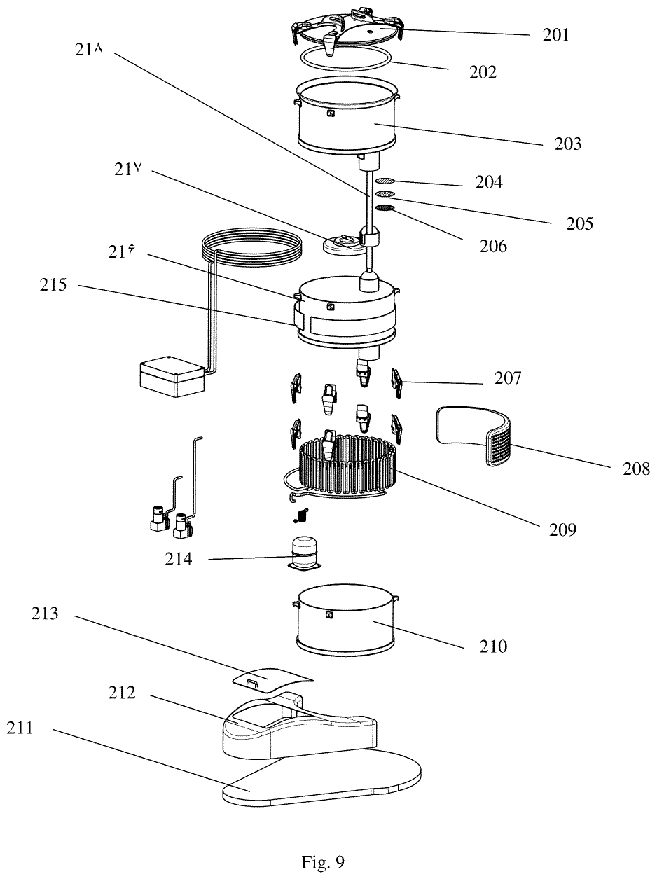

1. A reactor for the production of growth factors to regenerate the epithelium of the auditory system from embryonic stem cells, aimed at stimulating sensory tissue to enhance hearing capacity, in the form of a lyophilized powder includes at least three sections, several locks and levers for connecting the sections to each other, at least one UV lamp and at least several tube-shaped jackets and at least one thermal unit connected to the tubes and several micro-filters and at least one lever for opening and closing the filters and at least one rotating magnet and at least one magnetizable rotor and at least one vacuum pump and at least one refrigeration system and at least one concave chamber, and at least one chassis.

2. The reactor for the production of growth factors according to claim 1, wherein the reactor consists of three sections that are arranged in layers on top of each other, with the first section made of shatterproof glass (Pyrex) and the other sections made of stainless steel 316L.

3. The reactor for the production of growth factors according to claim 1, wherein tube-shaped jackets are installed around the glass vessel, that these tubes have inlets and outlets for the circulation of temperature-controlled water to maintain the internal temperature of the vessel at a constant 4° C.

4. The reactor for the production of growth factors according to claim 1, wherein, after transferring the required amount of umbilical cords from multiple embryos under a hood, the washing and sterilization process is performed using 70% ethanol, and the sterilized umbilical cords are then cut into smaller pieces.

5. The reactor for the production of growth factors according to claim 1, wherein for the cultivation of stem cells, the upper section of the reactor is initially filled with a culture medium composed of sterile fetal bovine serum (FBS) and DMEM.

6. The reactor for the production of growth factors according to claim 1, wherein enzymes such as trypsin and buffers such as phosphate-buffered saline (PBS) are added to the solution to create suitable conditions for cell culture.

7. The reactor for the production of growth factors according to claim 1, wherein in the serum of the culture medium, inhibitors of signaling pathways such as BMP are added, allowing stem cells to differentiate into epithelial pre-otic cells, which subsequently develop into sensory cells of the inner ear.

8. The reactor for the production of growth factors according to claim 1, wherein the GSK3 (glycogen synthase kinase 3) inhibitor aids in regulating the cell growth process.

9. The reactor for the production of growth factors according to claim 1, wherein fetal bovine serum is utilized due to its high levels of growth factors, abundant proteins, and ability to promote cell adhesion to the culture vessel surface.

10. The reactor for the production of growth factors according to claim 1, wherein, before placing the Wharton's jelly, the medium is prepared by adding sodium bicarbonate powder, and a very low concentration of HCL is used to adjust the pH to a range of 2-7 to 4-7.

11. The reactor for the production of growth factors according to claim 1, wherein after a period of 20 days to 1 month, by injecting PBS and sterile distilled water along with the enzyme trypsin, the glycoprotein bonds formed between the generated cells and the surface of the container are broken.

12. The reactor for the production of growth factors according to claim 1, wherein a micro-filter is installed in the outlet passage of the upper section, and by turning the lever 90 degrees to the right, the solution is directed from this section towards the filters and transferred to the middle section.

13. The reactor for the production of growth factors according to claim 1, wherein the presence of a covered magnet inside the upper compartment of the reactor and an electrically magnetizable rotor located between the upper and middle section continuously stirs the culture medium, resulting in the homogenization of the solution and the return of cells that have adhered to the walls of the vessel back into the solution.

14. The reactor for the production of growth factors according to claim 1, wherein in the middle section, a vacuum pump reduces the ambient pressure, leading to the evaporation of water present in the culture medium at temperatures below 100° C., which results in a higher concentration of beneficial substances in the solution.

15. The reactor for the production of growth factors according to claim 1, wherein, upon reaching the desired concentration, the concentrated solution is gradually passed through filters with very fine mesh, coated with nano-silver, by opening the lower valve of the second section, and subsequently enters the environment of the lower section.

16. The reactor for the production of growth factors according to claim 1, the lower section functions as a freeze-dryer designed to produce lyophilized powder from the stem cell conditional medium.

17. The reactor for the production of growth factors according to claim 1, the temperature of the lower section, which functions as a freezer, is reduced to −80° C., while simultaneously, the pressure in the lyophilized powder production section is decreased to 0.1 millibars and at the end of the process, this pressure is reduced to 0.03 millibars.
